(19) United States
(12) Patent Application Publication
(10) Pub. No.: US 2024/0142446 A1
(43) Pub. Date: May 2, 2024

(54) TEST KITS, DEVICES AND METHODS FOR DETECTING HIV INFECTION

(71) Applicant: MAXIM BIOMEDICAL, INC., ROCKVILLE, MD (US)

(72) Inventors: Mei-Jhy SU, North Potomac, MD (US); Juehn-Shin MAA, North Potomac, MD (US)

(73) Assignee: MAXIM BIOMEDICAL, INC., ROCKVILLE, MD (US)

(21) Appl. No.: 18/498,758

(22) Filed: Oct. 31, 2023

Related U.S. Application Data
(60) Provisional application No. 63/420,813, filed on Oct. 31, 2022.

(51) Int. Cl. G01N 33/543; G01N 33/569

(52) U.S. Cl. CPC G01N 33/54388; G01N 33/56988

(57) ABSTRACT

Devices, methods and test kits for the detection of Human Immunodeficiency Virus Type (HIV) infection are disclosed. The methods comprise the use of single use, qualitative, manually performed, visually read, in vitro immunoassays for the detection of antibodies to Human Immunodeficiency Virus Type 1 (HIV-1) and Type 2 (HIV-2); the immunoassays enable distinguishing between recent and long-term infection in HIV-1 infected subjects. The assay is a point of care (POC) test intended for use with blood or serum/plasma specimens.

### TECHNICAL FIELD

Embodiments of the present disclosure relate generally to test kits, devices and methods for detecting infection, such as the presence of one or more foreign components in bodily fluids. In particular, test kits, systems and methods comprising the detection of human immunodeficiency virus are provided.

### BACKGROUND OF THE INVENTION

The confluence of an urgent need for detecting infections, increasing health care costs together with increasing knowledge concerning the causality of many health conditions, mandates a prudent approach to monitoring factors that cause illness or lead to poor physiological status. The use of information concerning infectious agents and biomarkers may be incorporated into tests that allow individuals to follow their health and well-being and to make adjustments to their lifestyle (i.e. diet, exercise, quarantine) as necessary. Just as glucose monitors have been instrumental in enabling diabetic patients to monitor blood sugar levels and thereby manage their healthcare, there exists tremendous potential and need for tests that utilize other markers to better maintain health and control infection. The detection of infectious agents, antigens, biomarkers and the like, in bodily fluids can be useful in detecting disease, predicting risk, screening, diagnosis, scaling severity, monitoring progress, predicting response to therapy, determining prognosis, and understanding disease mechanism.

The rapid detection of infectious agents is especially important in the case of severe disease outbreaks such as during epidemics or pandemics. Under such scenarios, accurate and fast detection of an infection can facilitate not only early treatment, but also help to limit the spread of infection to multiple individuals, communities and even populations.

The Human Immunodeficiency Virus (HIV) is a retrovirus discovered in 1983 and is the etiologic agent for Acquired Immunodeficiency Syndrome (AIDS). Individuals infected with the virus see a depletion of T-helper cells, leaving patients susceptible to opportunistic infections and certain malignancies. The common routes of transmission are via sexual contact, exposure to contaminated blood and their associated products (such as contaminated syringes and needles), mother-to-newborn transmission, and tissue transplantation. There are two major types of HIV; Type 1 (HIV-1) and Type 2 (HIV-2). HIV-1 has been divided in to three major groups: group M (10 subtypes), group O and group N. Similarly, HIV-2 has been classified into at least 5 subtypes. Although there is some degree of immunological cross-reactivity between types and subtypes of HIV, the incorporation of type-specific protein sequences into a diagnostic assay design is thought to allow for reliable detection of antibodies to all groups and subtypes.

Diagnosis of HIV infection by detection of antibodies against viral proteins is well established and has clear and obvious benefits to individuals and the community. The CDC estimates that up to one-third of infected persons in the USA are unaware of their status, and this figure is almost certainly worse in less developed countries, so there is a clear need for improvement in testing rates. The global health community needs to know where HIV is spreading and where intervention is most effective in reducing the expansion of HIV. HIV-1 incidence assays can be used to detect recently infected persons and estimate the rate of HIV-1 infection. This information can be useful for identifying hot spots, conducting surveillance, intervention program planning, and vaccine and prevention trials.

The earliest laboratory-based incidence assays included less sensitive commercial HIV immunoassays¹⁻⁸ where the lower titers of anti-HIV antibodies typical of recent infections were used as a basis for identifying individuals likely to be recently infected. Since these desensitized commercial HIV immunoassays were developed by altering commercial assays that employ HIV-1 subtype B antigen(s), these assays were found to be less accurate in populations with primarily non-subtype B infections⁹⁻¹⁶.

The United States' CDC developed avidity assays, including a one-well avidity assay using limiting amounts of antigen¹⁷ with a new recombinant protein, rIDR-M, which contains the major variants of gp41 immunodominant regions amid the HIV-1 group M viruses. Well characterized samples were tested, and results indicated that subtype bias was minimized¹⁸⁻¹⁹.

Rapid testing has significant advantages over laboratory testing. Result receipt rates from lab tests may be as low as 50%, whereas virtually all rapid test results are relayed directly to the patient; rapid testing has also been shown to improve testing rates and linkage to care. Earlier linkage to care improves patient prognosis and reduces disease transmission. In addition, rapid testing of pregnant women can be carried out even during labor and still return results in time for pre-delivery antiretroviral therapy, which reliably and dramatically reduces mother-to-child transmission rates. Rapid testing is also vital to other time-sensitive applications such as emergency medicine and immediate testing after accidental exposure of healthcare workers.

The use of rapid test technologies has upstaged the use of classic ELISA and Western blot systems due to their increased ease of use and applicability over more diverse testing scenarios. ELISA and Western blot tests are feasible only when there is availability of highly trained manpower, instrumentation, and electricity. These tests are not often feasible, particularly in rural areas. If testing is to occur, samples must often be sent to remote cities where there are more advanced laboratories. It has been shown that possible combinations of highly sensitive and specific rapid test kits with adaptable field conditions can adequately be used to test for the presence of antibodies to HIV-1/2, thus eliminating the need for ELISA, WB, and PCR results.

What is needed therefore are efficient and rapid tests that enable the detection of infectious agents. Preferably, such tests should be in a patient-acceptable format, and be simple, safe, promoting the identification of greater numbers of HIV infected persons with the ultimate goals of early identification, early treatment and prevention of disease transmission. The characteristics of such a test, i.e. rapid, simple, accurate and safe, enhance the ability of public health programs, particularly in developing nations, to control and monitor HIV infections, both in conventional medical settings as well as in remote areas with limited access to conventional medical facilities or trained medical personnel.

### SUMMARY OF THE INVENTION

In an embodiment, the present disclosure relates to detection methods and devices for identifying HIV infection.

In an embodiment, the present disclosure relates to detection methods and devices comprising rapid test enabling the qualitative detection of antigens related to HIV infection.

In an embodiment, the present disclosure relates to HIV infection assays wherein the assays are single use, qualitative, manually performed, visually read, in vitro immunoassays for the detection of antibodies to Human Immunodeficiency Virus Type 1 (HIV-1) and Type 2 (HIV-2) and distinguish between recent and long-term infection in HIV-1. The assay comprises a point of care (POC) test intended for use with all biological specimens including saliva, blood, serum, plasma and urine specimens.

In an embodiment, the immunoassays of the disclosure comprise a single-use point-of-care immunoassay for verification of HIV diagnosis, (which classifies specimens as HIV positive or negative) and the classification of HIV-1 positive specimens as recent or long-term infections. In certain embodiments, results may be obtained in 1-45, 1-30, 1-20 minutes. In certain embodiments, the immunoassay comprises the following components: a device for specimen capture (such as a capillary transfer pipette/inoculation loop), running buffer in a capped sample tube, and a test cassette. The test strip with the cassette is composed of several materials which, in combination, are capable of detecting HIV antibodies and measuring antibody avidity.

In other embodiments, the present disclosure provides test kits comprising a device for collecting a biological sample (such as an inoculation loop or capillary transfer pipette), running/sample buffer tube in a capped sample tube, a test cassette, label sheet and instruction guide.

In certain embodiments, the present disclosure provides methods comprising the use of a device such as an inoculation loop, or pipette to obtain a sample from a subject requiring an assessment concerning HIV infection, utilizing the collected sample in a lateral flow assay and determining the presence of a HIV infection by the detection of a visual signal.

In other embodiments, the present disclosure provides uses of novel lateral flow assays enabling the visual detection of a signal to indicate the presence or absence of an HIV infection.

In certain embodiments, the present disclosure comprises the use of rIDR-M antigens in rapid and portable lateral flow assays for identifying recent and long-term HIV infections.

In certain embodiments, the novel recent infection assays (RIAs) disclosed herein are provided in the form of kits comprising the following features: (1) Component Processed Membrane/Analytical Region—the region in the lateral flow immunoassay for binding proteins at the test and control areas and to maintain their stability and activity over the shelf-life of the product; (2) Component Processed Sample Pad—for accepting the sample, treating it such that it is compatible with the assay, and releasing the analyte with efficiency; (3) Card Assembly—the wick, membrane, sample pad is laminated onto backing card (approximately 30 cm width). (This card is in turn cut into strips (approximately 4.8 mm)); (4) Pouching and Packaging—The cassette formatted strip is packaged into a two compartment Mylar pouch and heat sealed. Twenty (20) test set pouches are then packed into a Test Kit box, along with twenty (20) vials of running buffer, twenty-three (23) of 2 μL and 5 μL of capillary pipet respectively and product inserts.

### BRIEF DESCRIPTION OF FIGURES

FIG. 1 provides Table 1 listing various chemicals used in an embodiment of the lateral flow infection assays described herein. The table provides the chemicals by the test strip component on which they reside and an assessment of toxicology for each chemical is also provided. (Abbreviations: GRAS: Generally Recognized as Safe; PE: polyethylene; PET: polyester)

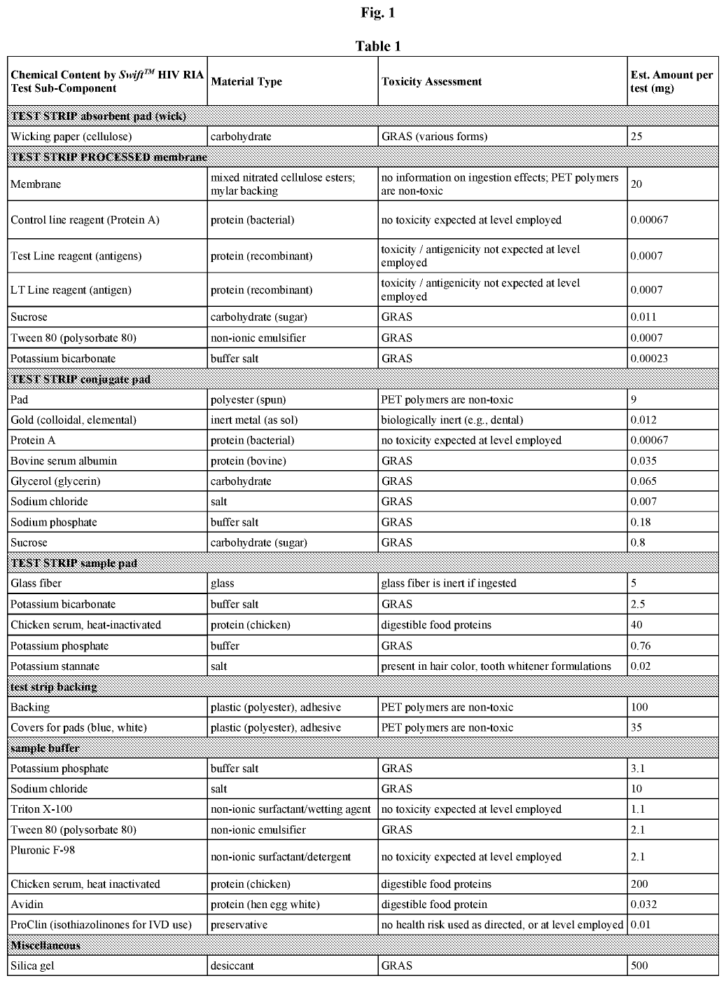

FIG. 2 provides a schematic in exploded view of components constituting an embodiment of the lateral flow infection assay showing arrangement on an assembled card.

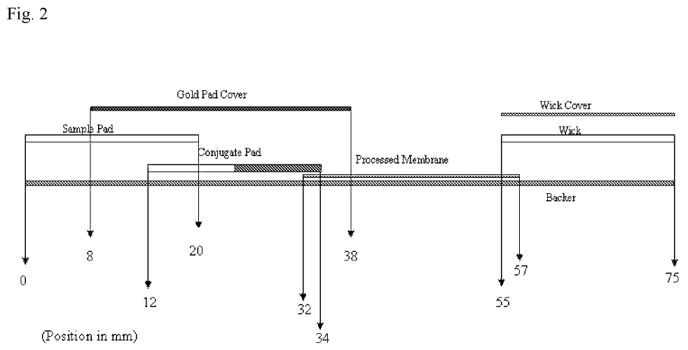

FIG. 3 provides a schematic showing an embodiment of cassette housing for the lateral flow assays described herein.

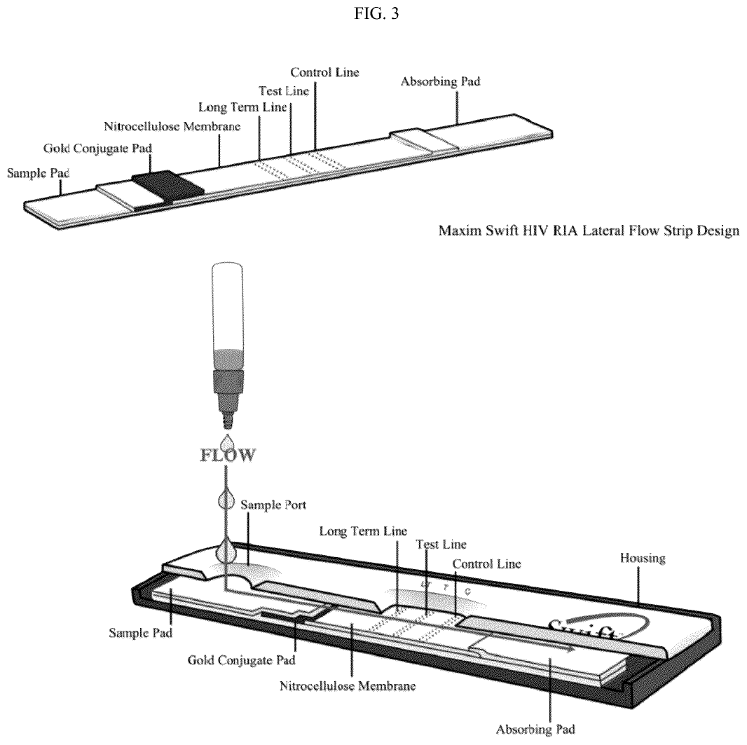

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety including U.S. Provisional Patent Application Ser. No. 63/420,813 filed on Oct. 31, 2022.

The term “subject” should be construed to include subjects, for example medical or surgical subjects, such as humans and other animals suffering from viral infection.

The human immunodeficiency viruses (HIV) are two species of Lentivirus (a subgroup of retrovirus) that infect humans. Over time, they cause acquired immunodeficiency syndrome (AIDS), a condition in which progressive failure of the immune system allows life-threatening opportunistic infections and cancers to thrive. Without treatment, average survival time after infection with HIV is estimated to be 9 to 11 years, depending on the HIV subtype.

According to HIV.org, an estimated 1.3 million individuals worldwide acquired HIV in 2022, women and girls accounted for 46% of all new HIV infections in 2022. Approximately 86% of people with HIV globally knew their HIV status in 2022. The remaining 14% (about 5.5 million people) did not know they had HIV and still needed access to HIV testing services. The World Health Organization estimates that in 2022, approximately 630,000 [480,000-880,000] people died from HIV-related causes globally. Since 2010, HIV-related deaths have been reduced by 51%, from 1.3 million [970,000-1.8 million]. The global HIV epidemic claimed 69% fewer lives in 2022 since the peak in 2004 and approximately 84,000 [56,000-120,000] children died from HIV-related causes in 2022. Despite the reduction in incidence and death, HIV continues to be a major global public health issue, claiming 40.4 million [32.9-51.3 million] lives so far. HIV testing is an essential gateway to HIV prevention, treatment, care, and support services.

Viral isolation and a number of methods for detection of viral antigens, nucleic acids, and antibodies (serology) are the fundamental techniques used for the laboratory diagnosis of viral infections. Viral isolation by means of cell culture is virtually always performed in designated virology laboratories. Other methods may be performed in those laboratories as well but may also be performed in diverse laboratory sections such as general microbiology, serology, blood bank, clinical chemistry, pathology, or molecular virology. In the case of HIV, there is a serious and urgent need for diagnostic testing to be done outside of traditional laboratories with a need for rapid, easy-to-use testing in locations such as homes, schools, and businesses without the need for laboratory processing. One of the most commonly used detection assays for viral antigens involves the use of enzyme-linked immunoassays (EIAs). EIAs are available in many formats, including for example lateral flow immunoassays. Lateral flow immunoassays may also be referred to as lateral flow tests (LFT), lateral flow devices (LFD), lateral flow assays (LFA), lateral flow immunoassays (LFIA), lateral flow immunochromatographic assays, dipstick tests, express tests, pen-side tests, quick tests, rapid tests, test strips. As used herein, lateral flow immunoassays are intended to include each of the preceding terms and other such terms known to those skilled in the art.

Lateral flow immunoassays (LFIAs) are typically simple to use diagnostic devices used to confirm the presence or absence of a target analyte, such as pathogens or biomarkers in humans or animals, or contaminants in water supplies, foodstuffs, or animal feeds. LFIAs typically contain a control line to confirm the test is working properly, along with one or more target or test lines. They are designed to incorporate intuitive user protocols and require minimal training to operate. They can be qualitative and read visually, or provide data when combined with reader technology. Lateral flow tests are widely used in human health for point of care testing. They can be performed by a healthcare professional or by the patient, and in a range of settings including the laboratory, clinic or home. In the medical diagnostic industry, there are strict regulatory requirements which must be adhered to for all products developed and manufactured. LFIAs generally use immunoassay technology comprising the use of nitrocellulose membranes, colored nanoparticles, and antibodies to generate results.

In an embodiment of the invention, nanoparticles are selected based on their conjugation properties to both the pad and the antibody/antibodies being utilized to capture the analyte. Suitable nanoparticles include those constructed from colloidal gold, latex and cellulose and they may be present in a variety of shapes such as, but not limited to, spheres, beads, or rods. The size range of the nanoparticles varies from 20 nm-400 nm. In certain embodiments, colloidal gold particles are used. In certain other embodiments, latex labels which can be tagged with a variety of detector reagents such as colored or fluorescent dyes, and magnetic or paramagnetic components are used. As latex can be produced in multiple colors, it has an application in multiplex assays, which require discrimination between numerous lines. Carbon and fluorescent labels, or enzymatic modification of the labels, may also be used to improve the sensitivity of the assay. In certain embodiments nanoparticles constructed of cellulose are used. The lateral flow assay technology utilized resulted in the selection of nanoparticles that generate a visually detectable signal by the eye that can be used with or without a reader for interpretation.

Conjugation of the antibody or antigen to the nanoparticle comprises specific consideration of using covalent or passive forces and techniques to bind an antibody (or antigen) to a nanoparticle. In certain embodiments, a “passive” technique is used, involving the optimization of antibody and particle ratio. In certain embodiments, a “covalent” technique is used, involving the optimization of antibody/particle ratio and EDC/NHS ratios. In certain embodiments for NHS, the antibody to nanoparticle ratio comprises 0.6:1, 0.8:1, 1:1, 1.2:1, or 1.3:1. In certain embodiments for EDC, the antibody to nanoparticle ratio comprises 1:500, 1:750, 1:1000, 1:1500, or 1:2000.

In an embodiment, the aspect of “target detection”, referring to the method of embedding the biological materials (i.e. antibody/antigen/nanoparticles/other chemicals) on a test strip is taken into consideration. One option under this consideration is “Line vs. Spot” wherein capture the antibody/antigen can be deposited on a membrane in the form of a line or a spot. Another option for target detection comprises “Singlex vs. Multiplex” referring to the number of biological targets being detected on a test strip.

The present disclosure provides a novel test for the detection of HIV-1 positive specimens as recent or long-term infections and is also referred to herein as the Maxim Swift™ HIV RIA. The test kit comprises a single-use, point-of-care, chromatographic immunoassay for verification of HIV diagnosis. Results can be obtained within 1-45, 1-30 or 1-20 minutes. The Maxim Swift™ HIV RIA is comprised of a device for specimen capture (such as a capillary transfer pipette/inoculation loop), running buffer in a capped sample tube, and a test cassette. The test strip with the cassette is composed of several materials which, in combination, are capable of detecting HIV antibodies and measuring antibody avidity.

Additional materials useful for utilizing the tests described herein include the following: timer, personal protection equipment, and biohazardous waste containers. In an embodiment, the test kits are stored at 4-30° C. until the stated product expiration date and users are advised to allow the test kits to come to room temperature (15-30° C.) before using.

The test kits as described and claimed herein may further comprise a listing of instructions for using the test kit and for interpreting the results thereof. The listing of instructions provides details concerning the collection of a bodily fluid, the introduction of the bodily fluid to the test kit, the application of the bodily fluid to the lateral flow assay, operation of the test kit, interpretation of results, and effective disposal of the device.

In addition, the test kits of the invention further comprise packaging for disposing the test kit, and the packaging may be configured to be compliant with requirements for disposing biohazardous materials.

In certain embodiments the bodily fluids suitable for use with the test kits described herein comprise saliva, sputum, tears, sweat, mucus, serum, semen, urine and blood, to detect biomarkers, including but not limited to, analytes, metabolites, chemicals, hormones, toxins, enzymes, immunoglobulins, proteins, and nucleic acids.

In certain embodiments, the reagents necessary for performing a detection assay include, but are not limited to, bovine serum albumin, urea, detergents, emulsifiers, surfactants, non-ionic surfactants, Triton X-100, sucrose, methanol, buffers, bovine serum albumin blocking buffer, polysorbate 20, Tween 20, polysorbate 80, Tween 80, biocides, ProClin™, sodium chloride, sodium phosphate monobasic, sodium phosphate dibasic, and heptahydrate.

Maxim Swift™ HIV RIA Test

Provided herein are single-use point-of-care immunoassays for verification of HIV diagnosis, (classifying specimens as HIV positive or negative) and the classification of HIV-1 positive specimens as recent or long-term infections (the test may be referred to as the Maxim Swift™ HIV Recent Infection Assay (RIA) test). In certain embodiments, the results are obtained rapidly, from 1-45, 1-30 or 1-20 minutes. The immunoassay test kit comprises a device for collecting a biological sample (such as an inoculation loop or capillary transfer pipette), running buffer in a capped sample tube, and a test cassette. The test strip with the cassette is composed of several materials, including rIDR-M antigens, which, in combination, are capable of detecting HIV antibodies and measuring antibody avidity.

In an embodiment, the specimen may be collected as serum, plasma, or blood by conventional clinical procedures such as venipuncture, lancet finger-stick, and serum or plasma separation. Using the provided inoculation loop or capillary transfer pipette, the specimen is transferred into the provided running buffer. This mixed sample is then added into the cassette's sample well and absorbed into the test's sample pad to initiate the test run. This sample pad contains conditions the specimen and prepares it for optimal reactivity through the remainder of the test strip. The sample mixture continues to migrate up the test strip by a wicking action, until it rehydrates the protein A gold conjugate. The protein A gold conjugate confers a reddish-purple to purplish-gray coloration which will bind to both HIV-positive (if present) and HIV negative antibodies in the specimen liquid.

The colored liquid will continue to move up the Test Strip onto the nitrocellulose membrane which contains three reagent lines (in order of sample contact: Long Term Line [marked “LT” position], Test Line [marked “T” position] and Control Line [marked “C” position]). Once the liquid mixture starts to migrate onto the membrane, the user will see a reddish-purple to purplish-gray liquid mixture migrate up the Test Strip in the test window. The liquid will continue to be drawn up to the absorbent pad of the Test Strip until the color on the membrane has cleared, approximately 1-20 minutes after the start of the test.

As the liquid containing antibodies bound to the conjugate crosses the membrane, it first encounters the LT Line, which contains the HIV-1 rIDR-M recombinant antigen. The liquid specimen continues to migrate up the Test Strip, next encountering the Test Line, which contains gp41 and gp36 recombinant antigens which will bind any HIV-1 and HIV-2 antibodies present in the specimen. Finally, the liquid specimen will continue to migrate up the strip, encountering the Control Line. The Control will bind human antibodies in the liquid regardless of whether those antibodies are HIV positive or negative. If an adequate sample has been collected and the test is run correctly, antibodies will be present in the specimen, and will have bound to the conjugate and captured on the Control Line, giving a visible reddish-purple line. In all cases, the color intensities of the Control Line, the Test Line and the Long-Term Line, do not necessarily correlate to the amount of antibody captured. The appearance or non-appearance of each line is determined by the number of antibodies detected. The results of the test are interpreted at approximately 1-45, 1-30, or around 20 minutes. At this time, the antibodies will have had adequate time to migrate up the entire Test Strip encountering both the colored protein A-gold colloid conjugate and the three reaction lines to give a test result.

Assay Components

(a) Protein A-Colloidal Gold Conjugate and Gold Conjugate Pad

Protein A-Colloidal Gold conjugate is prepared by reducing gold chloride with sodium citrate to obtain colloidal gold sol. The gold sol quality is monitored by absorbance at the peak wavelength. Protein A is mixed with the colloidal gold at the proper proportions. The resulting Protein A-colloidal gold conjugate is concentrated and by absorbance at the peak wavelength. Protein A-Colloidal Gold Conjugate of appropriate concentration is striped onto a glass fiber pad and dried. This gold conjugate pad intermediate is assembled into a “card” containing the gold conjugate pad, striped nitrocellulose pad (with antigen and goat anti-human IgG) and absorbent pad. This card is cut into individual test strips. The built card is tested with an internal QC (quality control) panel.

(b) Long Term Line (LT) (Membrane Signal Reagent)

Signal reagent is prepared as an aqueous solution of recombinant HIV-1 antigens of appropriate concentration (approximately 25-250 ug/ml). In an embodiment, the antigens comprise rIDR-M. The signal reagent is striped onto the nitrocellulose membrane in the LT line and dried. Membrane striped with the antigens is tested functionally in a sample preparation using known standards as described in the Membrane Striping section below.

(c) Test Line (T) (Membrane Signal Reagent)

Signal reagent is prepared as an aqueous solution of two recombinant HIV antigens of appropriate concentration (approximately 0.05-3.0 mg/ml). In an embodiment, the antigens comprise gp41 and gp36. The signal reagent is striped onto the nitrocellulose membrane in the test line and dried. Membrane striped with the antigens is tested functionally in a sample preparation using known standards as described in the Membrane Striping section below.

(d) Control Line (C) (Membrane Control Reagent)

Control reagent is prepared as an aqueous solution of goat anti-human antibody/Protein A of appropriate concentration (approximately 0.2-1.5 mg/ml). The control reagent is striped onto the nitrocellulose membrane in the control line and dried. Membrane striped with the goat antibody/Protein A is tested functionally in a sample preparation using known standards as described in the Membrane Striping section below.

(e) Membrane Striping

An approved lot of signal reagent and control reagent using currently accepted membrane is used to prepare membrane with (rIDm antigen, HIV1/2 antigens and control) striping levels at 0.04-0.08 μL/mm. Pre-qualification cards are assembled with a matched gold conjugate pad and tested to determine the striping concentration. Striping concentration is tested for functionality against internal QC standards. Based on this prequalification, the full lot of membrane is striped at the approved striping level with all of signal and control reagents dried.

Striping procedures are performed in controlled environmental conditions. An approved lot of signal reagent and control reagent using currently accepted membrane is used to prepare membrane with (antigen and control) striping levels at 0.04 μL/mm, 0.05 μL/mm, 0.06 μL/mm, 0.07 μL/mm, and 0.08 μL/mm. Pre-qualification cards are assembled with a matched gold conjugate pad and tested to determine the striping concentration. Striping concentration is tested for functionality against internal QC standards. Based on this prequalification, the full lot of membrane is striped at the approved striping level with both signal and control reagents dried.

(f) Sample Pad Preparation

In an embodiment, the sample pads of the lateral assays use glass fiber pads treated with a blocking buffer containing protein, detergents and salts. Impregnated sample pads are dried overnight and stored under desiccation at 15-30° C. Sample pads are qualified at Card Assembly, in a manner similar to Gold Conjugate Pad and Striped Membrane. Sample pads are tested against internal QC standards.

(g) Card Assembly

Card assembly is performed in controlled environmental conditions. Components required to assemble cards are 1) Absorbing Pad/Wick, 2) striped processed membrane, 3) gold conjugate pad (a laminate covering the gold conjugate solely for aesthetic and product identity purposes), 4) sample pad and 5) backing card. Assembled cards are inspected and tested for functionality against internal QC standards.

Strip cutting and pouching is performed in controlled environmental conditions. Assembled cards are cut into approximately 4.8 mm wide strips. Strips are placed into foil pouches with desiccant and a 12×75 mm polystyrene test tube and sealed with a heat sealer (the test tube was packaged as a separate item). The foil pouch has lot number and expiration date information printed on it prior to pouching.

(h) Description of Specimen Collection and Transport Materials Provided

In certain embodiments, the infection assay systems of the disclosure comprise: one a device for specimen capture (such as a capillary transfer pipette/inoculation loop, i.e. transfer pipette) (2 μL, for serum/plasma sample and 5 μL for blood sample)), and one specimen collection tube with sample buffer (400 μL). In certain embodiments, the buffer comprises 10% protein serum, 3.2% surfactants, 0.3% anti-bacterial/fungi reagents with 2.5% combined salts

(i) Kit Format

In certain embodiments, the infection assay of the disclosure is provided in a kit format. The kit format for one individual kit comprises the following:
a. One (1) Package Insert written in English
b. Twenty (20) Individually Foil Pouched Test Cassette-strips with desiccant (Major Subcomponent, PN-81300) labeled with Product Name “Maxim Swift HIV RIA”, Lot Number, and Expiration Date
c. Twenty-three (23) 2 μL of Capillary Transfer Pipettes (or other collection device)
d. Twenty-three (23) 5 μL of Capillary Transfer Pipettes (or other collection device)
e. Twenty (20) filled Sample Buffer Tubes

In certain embodiments of the disclosure, the novel assays described herein utilize technology related to a one-well avidity assays using limiting amounts of antigen with a new recombinant protein, rIDR-M, which contains the major variants of gp41 immunodominant regions amid the HIV-1 group M viruses. This technology has been uniquely refined and engineered for novel features enabling the development of a rapid infection assay comprising the incorporation of rIDR-M antigen to identify recent and long-term infections, but in a portable, rapid lateral flow type of format.

Design and Manufacturing Information

(a) Sample Buffer and Tube

After sample collection, the sample collection device is immersed into a sample buffer and mixed with the buffer. The buffer (diluent) is of an appropriate volume and contains an antimicrobial agent to minimize microbial degradation. The diluent also renders the sample easily pipettable, and aesthetically non-objectionable to non-laboratory trained personnel. The diluent serves to extend sample volume so that sample will remain after initial testing, which makes possible repeat testing of possible invalid tests, confirmation testing and QC/QA programs based on actual rather than contrived specimens. The diluent pH and its protein, salt and detergent content was specifically designed to be compatible with a wide range of ELISA and western blot systems as well as the lateral flow immunochromatography strip used in the rapid infection test of the disclosure. In certain embodiments, the diluent comprises approximately 5-10% Tween 80, 2.5-10% PEG with 4.4% bicarbonate/phosphate salts.

The sample buffer tube is comprised of an inert plastic that neither releases chemically active compounds nor binds significantly to sample analytes; it has a closure cap capable of preventing evaporation over the expected shelf life of the kit. Polypropylene is well established as a material that meets the first two criteria. In certain embodiments, the closure cap is chosen from a range of commercially available options by accelerated evaporation testing in heated incubators, confirmed by real-time testing under normal storage and use conditions. The screw cap selected is made of low density polyethylene, a well-established inert and safe material, it also features an O-ring design (or other equivalent) for providing a superior seal.

(b) Antigens (Test Line of Processed Membrane)

In an embodiment, the recent infection assays of the disclosure involve an optimized process for antigen striping onto the assay membrane. First, the nature of the antigen and the number of total available antigenic sites is optimized. Such considerations were evaluated in the general context of the desired immunochemical event (‘signal’ on a strip), the specific nature of the desired signal (generation of the immuno-complex and its immobilization), and the characteristics and limitations of the testing format. The inventors herein designed the assays based on the goal of selecting either intact proteins (e.g., the characteristically highly antigenic gp41 protein of HIV-1) or other appropriately antigenic peptides. Intact proteins (either in viral lysates or purified proteins) have historically been subject to cross-reactivity of sample antibodies to non-viral proteins in the antigen preparation, even under the best of purification regimens. Also, by specifying recombinant proteins or synthetic peptides, the inventors were able to avoid infectious reagent issues; these pose no risk of infection since no virus particles are used. We focused our evaluation on several peptides and several recombinant proteins.

Certain embodiments of the novel lateral assays comprise the use of synthetic peptides as antigens. The antigens exhibit excellent reactivity in the prototype Maxim Swift™ HIV Recent Infection Assay rapid test format. The HIV-2 peptide (representing gp36, the HIV-2 analog of gp41) performed well in the detection of HIV-2 specimens, showing little or no cross-reactivity with HIV-1 (only) specimens, and is compatible with the HIV-1 peptide. Thus it can be applied together with the HIV-1 peptide, in the same line on the membrane. In certain embodiments, peptides utilized in the assays are sourced from Fapon Biotech (Guangdong, China).

Additional aspects of these peptides make them desirable as detection reagents: they show reactivity which is typically equivalent to, or better than recombinant antigens evaluated; although they cannot be readily bound to nitrocellulose membrane directly, they can be easily conjugated to avidin or streptavidin via an N-terminal biotin in one commercially available form to provide a mechanism to anchor them to the membrane surface; they can be applied in a more concentrated form than many other antigens, allowing for a narrower reagent line; and there is less lot-to-lot variability and fewer contaminants for synthetic peptides than for both recombinant proteins (which require growing the host organism, releasing proteins from it, then purifying the expressed recombinant from a complex mixture) and native proteins (which also require purification but from a much more complex mixture of proteins).

An additional advantageous feature of the invention is that that the HIV-2 recombinant antigen analogous to the HIV-1 antigen gp41, the gp36 envelope protein, added to the system do not adversely affect either the sensitivity or specificity of the test. With the rejection of the recombinant gp160 and the p24, selected the gp41/36 ortholog pair as described above.

(c) Goat Anti-Human Antibody, F(ab′)2/Protein A (Control Line)

As known to those skilled in the art, several options for control line systems for use in the assay are available. For an embodiment, Protein A and animal antibodies specific to the heavy chain (F(c)) of human antibodies were tested. Protein A is a protein that binds to the heavy chain (F(c)) of human antibodies and may be utilized for certain assays.

In another embodiment, goat anti-human antibodies are selected as a control line. Both Kirkegaard and Perry Laboratories (KPL) (Gaithersburg, MD, USA) and Jackson Immunoresearch (West Grove, PA, USA) both provide a F(ab′)2 Goat Anti-Human F(c). Both systems were tested in the platform and were found to be equivalent.

(d) Nitrocellulose Membrane

In an embodiment, Millipore SPHF 180 (Merck KGaA, Germany) comprises the primary membrane for the lateral assay systems described herein. The membranes comprise cellulose ester and enable capillary flow. Additional membranes considered for use include Millipore SPHF 135, Schleicher & Schuell (S&S) FF125, and S&S AE99 (Whatman/GE Healthcare/Cytiva USA) and equivalents thereof.

(e) Conjugate Pad

In an embodiment, pretreated non-woven polyester (such as that available from (Whatman/GE Healthcare/Cytiva USA) comprises the conjugate pad material. Additional materials considered and suitable for use include glass fiber, and in-house treated non-woven polyester.

### REFERENCES

1. Centers for Disease Control and Prevention (CDC) Universal Precautions for prevention of transmission of human immunodeficiency virus, hepatitis B virus, and other bloodborne pathogens in health-care settings. MMWR 1988; 37(24):377-388.
2. Kanki, P. J., Hopper, J. R. and Essex, M. (1987) The origins of HIV-1 and HTLV-4/HIV/2. Ann NY Acad Sci 511: 370-375.
3. Nicoll, A., Gill, O. N. (1999) The global impact of HIV infection and disease. Commun Dis Publ Health 2: 85-95.
4. Valdiserri R. O., Holtgrave, D. R., West, G. R. (1999) Promoting early diagnosis and entry into care. AIDS 13: 2317-2330
5. Gallo R C, Salahuddin S Z, Popovic M, et al: Frequent detection and isolation of cytopathic retroviruses (HTLV-III) from patients with AIDS and at risk for AIDS. Science 224:500-503, 1984
6. Janssens W, Buvé A, Nkengasong J N: The puzzle of HIV-1 subtypes in Africa. AIDS 11:705-712, 1997. 21.
7. Charneau P, Borman A M, Quillant C, et al: Isolation and envelope sequence of a highly divergent HIV-1 isolate: definition of a new HIV-1 group. Virology 205:247-253, 1994.
8. Simon F, Mauclere P, Rogues P, et al: Identification of a new human immunodeficiency virus type 1 distinct from group M and group 0. Nature Medicine 4:1032-1037, 1998.
9. Janssen R S, Satten G A, Stramer S L, et al. New testing strategy to detect early HIV-1 infection for use in incidence estimates and for clinical and prevention purposes. JAMA 1998, 280:42-48 [Erratum JAMA 1999, 281:1893]
10. Kothe D, Byers R H, Caudill S P, et al. Performance characteristics of a new less sensitive HIV-1 enzyme immunoassay for use in estimating HIV seroincidence. J Acquir Immune Defic Syndr 2003, 33:625-634.
11. Diza R S, Kallas E G, Castelo A, Rawal B D, and Busch M P. Use of a new ‘less-sensitive enzyme immunoassay’ testing strategy to identify recently infected persons in a Brazilian prison: Estimation of incidence and epidemiological tracing. AIDS 1999, 13:1417-1418.
12. McFarland W, Busch M P, Kellogg T A, et al. Detection of early HIV infection and estimation of incidence using a sensitive/less-sensitive enzyme immunoassay testing strategy at anonymous counseling and testing sites in San Francisco. J Acquir Immune Defic Syndr 1999, 22:484-489.
13. Sill A M, Kreisel K, Deeds B G et al. Calibration and validation of an oral fluid-based sensitive/lesssensitive assay to distinguish recent from established HIV-1 infections. J Clin Lab Anal 2007. 21:40-45.
14. Constantine N T, Sill A M, Jack N, et al. Improved classification of recent HIV-1 infection by employing a two-stage sensitive/less-sensitive test strategy. J Acquir Immune Defic Syndr 2003, 32:94-103.
15. Young C L, Hu D J, Byers R, et al. Evaluation of a sensitive/less sensitive testing algorithm using the bioMerieux Vironostika-LS assay for detecting recent HIV-1 subtype B′ or E infection in Thailand. AIDS Res Hum Retroviruse 2003, 19:481-486.
16. Parekh B S, Hu D J, Vanichseni S, et al. Evaluation of a sensitive/less-sensitive testing algorithm using the 3A11-LS assay for detecting recent HIV seroconversion, among individuals with HIV-1 subtype B or E infection in Thailand. AIDS Res Hum Retroviruses 2001, 17:453-458.
17. Parekh B S, Kennedy M S, Dobbs T, et al. Quantitative detection of increasing HIV type 1 antibodies after seroconversion: a simple assay for detecting recent HIV infection and estimating incidence. AIDS Res Hum Retroviruses 2002, 18:295-307
18. Diza R S, Kallas E G, Castelo A, Rawal B D, and Busch M P. Use of a new ‘less-sensitive enzyme immunoassay’ testing strategy to identify recently infected persons in a Brazilian prison: Estimation of incidence and epidemiological tracing. AIDS 1999, 13:1417-1418.
19. McFarland W, Busch M P, Kellogg T A, et al. Detection of early HIV infection and estimation of incidence using a sensitive/less-sensitive enzyme immunoassay testing strategy at anonymous counseling and testing sites in San Francisco. J Acquir Immune Defic Syndr 1999, 22:484-489.

1. A test kit for distinguishing between recent and late stage human immunodeficiency virus infection in a bodily fluid, the test kit comprising:
a) a sample collection device,
b) a vessel containing sample/running buffer,
c) a cassette comprising an absorbing/wicking pad, striped processed membrane, conjugate pad sample pad, and backing card
d) instructions for use.

2. The test kit of claim 1, wherein the sample collection device comprises an inoculation loop, capillary transfer pipette, absorbent pad, swab, nasopharyngeal swab, sponge or pipette.

3. The test kit of claim 1, wherein the vessel containing running sample/running buffer, comprises an ampoule, bottle or sachet.

4. The test kit of claim 1, wherein the striped processed membrane comprises a nitrocellulose membrane comprising three reagent lines (in order of sample contact: Long Term Line [marked “LT” position], Test Line [marked “T” position] and Control Line [marked “C” position].

5. The test kit of claim 4, wherein the LT Line, comprises HIV-1 rIDR-M recombinant antigens.

6. The test kit of claim 5, wherein Test Line, comprises gp41 and gp36 recombinant antigens.

7. The test kit of claim 4, wherein the Control Line comprises goat anti-human antibodies.

8. The test kit of claim 7, wherein the sample pad comprises glass fiber pads treated with a blocking buffer containing protein, detergents and salts.

9. The test kit of claim 1, wherein the conjugate pad comprises Protein A-Colloidal Gold.

10. The test kit of claim 1, wherein the cassette comprises a lateral flow assay.

11. The test kit of claim 10, wherein the lateral flow assay is a point of care test.

12. The test kit of claim 1, wherein test results are obtained in 1-45, 1-30 or 1-20 minutes.

13. The test of claim 1, wherein the instructions for use provide details concerning the collection of a bodily fluid, the introduction of the bodily fluid to the test kit, the application of the bodily fluid to the cassette, operation of the test kit, interpretation of results, and effective disposal of the device.

14. The test kit of claim 1, further comprising packaging for disposing the test kit.

15. A method for distinguishing between recent and late stage human immunodeficiency virus infection in a bodily fluid comprising the use of a point of care lateral flow assay, wherein the lateral flow assay comprises an absorbing/wicking pad, striped processed membrane, conjugate pad sample pad, and backing card.

16. The method of claim 15, wherein the striped processed membrane comprises a nitrocellulose membrane comprising three reagent lines (in order of sample contact: Long Term Line [marked “LT” position], Test Line [marked “T” position] and Control Line [marked “C” position].

17. The method of claim 16, wherein the LT Line, comprises HIV-1 rIDR-M recombinant antigens, wherein Test Line, comprises gp41 and gp36 recombinant antigens and wherein the Control Line comprises goat anti-human antibodies.

18. The method of claim 17, wherein test results are obtained in 1-45, 1-30 or 1-20 minutes.

19. The method of claim 15, wherein the lateral flow assay is provided as a test kit.

20. The method of claim 19, wherein the test kit further comprises a sample collection device, a vessel containing sample/running buffer, and instructions for use.
